(19) United States
(12) Patent Application Publication
(10) Pub. No.: US 2010/0136677 A1
(43) Pub. Date: Jun. 3, 2010

(54) Apheresis Tubing Set

(76) Inventor: Glen James Wicks Delaronde-Wilton, Cardiff (GB)

Correspondence Address: K&L Gates LLP, STATE STREET FINANCIAL CENTER, One Lincoln Street, BOSTON, MA, 02111-2950, US

(21) Appl. No.: 12/506,557

(22) Filed: Jul. 21, 2009

Related U.S. Application Data
(63) Continuation of application No. 11/662,354, filed on Mar. 8, 2007, now abandoned.

(30) Foreign Application Priority Data
Sep. 9, 2004 (GB) 0419980.8
Sep. 29, 2004 (GB) 0421585.1

(51) Int. Cl. C12M 3/00

(52) U.S. Cl. 435/307.1

(57) ABSTRACT

An apheresis tubing set comprises a cryocyte bag for collecting cells separated during apheresis. The cryocyte bag may comprise a mixing compartment in fluid communication with a cell storage compartment, wherein the mixing compartment comprises a cryoprotectant port and a cell sample port and wherein the storage and mixing compartments are in fluid communication via a mix conduit. The cryocyte bag may comprise two or more independent cell storage compartments for collecting two or more aliquots of the cells

### CROSS-REFERENCE TO RELATED APPLICATIONS

This application is a continuation of U.S. patent application Ser. No. 11/662,354, filed Mar. 8, 2007, which is the U.S. national stage of International patent application number PCT/GB2005/003429, filed Sep. 7, 2005, which claims priority to and the benefit of Great Britain patent application number 0419980.8, filed Sep. 9, 2004, and Great Britain patent application number 0421585.1, filed Sep. 29, 2004, the contents of each of which are incorporated herein by reference.

### FIELD OF THE INVENTION

The invention relates to apheresis tubing sets, and in particular to apheresis tubing sets comprising a cryocyte bag for collecting cells separated during apheresis and to apheresis tubing sets which comprise a cell collect bag having two or more independent cell storage compartments. The invention also relates to leukapheresis tubing sets, and in particular to leukapheresis tubing sets which comprise a leukocyte collect bag having two or more independent leukocyte storage compartments.

### BACKGROUND TO THE INVENTION

### Apheresis and Leukapheresis

Apheresis is a procedure which involves the extraction from a patient of blood (or other component tissue of the hematopoietic system, such as bone marrow), the selective removal and retention of one or more fractions thereof (for example, plasma, leukocytes, erythrocytes, stem cells, platelets etc.) and the return of the remainder to the patient.

Leukapheresis is a specific form of apheresis which involves the selective separation and removal of leukocytes from withdrawn blood, the remainder of the blood then being retransfused into the donor. During leukapheresis, the removed blood is passed through a cell separation device which separates nucleated white blood cells from red blood cells and plasma outside the body. The red blood cells and plasma are returned to the individual, as part of the separation process. The process is essentially continuous, with blood being removed and returned almost simultaneously after various extractions have been performed.

Leukapheresis therefore makes it possible to remove and return the entire blood volume of the individual several times over and separate out and keep large numbers of white cells without detriment to the individual. The technique therefore relies on the establishment of a vein-to-vein extracorporeal blood circulation and extraction of leukocytes from the recirculating blood.

Aphereses are generally automated, and conducted using either continuous or interrupted flow centrifugation or filtration techniques, as described in “Leukapheresis and Granulocyte Transfusions”, published by American Association of Blood Banks, Washington D.C. (1975).

### Leukapheresis Devices and Tubing Sets

Many different types of leukapheresis devices are presently commercially available. Such devices usually comprise at least three separate elements: (1) a separation device (e.g. comprising a membrane or centrifuge rotor, which provides the forces for separating the leukocytes from the various other blood components; (2) one or more pumps for conveying the blood sample to the separation device, for removing the separated leukocytes and for maintaining the forces necessary for transfusion and retransfusion, and (3) a (normally disposable) tubing set which holds the blood and its various fractions in a particular geometry within the separation device, defines fixed channels through which the blood flows (normally in a circuit from the donor, through the leukapheresis device and back to the donor) as well as vessels (usually bags) for the collection of the separated leukocytes and/or other blood fractions or fluids. The various vessels usually take the form of flexible, transparent bags. Some (or all) of the tubing is usually formed of flexible, transparent material (e.g. plastics such as PVC).

The tubing set generally also includes a blood processing vessel within which the leukocytes are subjected to separation forces in the separation device. In the case of tubing sets adapted for use with centrifuge-based separation devices, the blood processing vessel may take the form of a centrifuge loop which defines a vessel within which the blood is subjected to centrifugal separation forces when loaded into the centrifuge rotor (usually in a channel in the centrifuge rotor) of the separation device. An example of such a tubing set is the COBE Spectra™ Automated Peripheral Blood Stem Cell Set (AutoPBSC Set). Alternatively, the blood processing vessel in such apparatus may take the form of a Latham bowl (for example, in the Haemonetics V50, see infra) or some form of filtration device.

The tubing sets may be closed, functionally closed, or open. The term closed system, as applied to a leukapheresis tubing set, is used to define tubing sets which are sterile and isolated from the outside environment by aseptic barrier(s) and in which all components are fully integral, being attached and/or assembled at the manufacturing site. The term functionally closed system, as applied to a leukapheresis tubing set, is used to define tubing sets which are assembled at the device manufacturing site and which use sterile barrier filters (e.g. sub-micron filters such as 0.22 micron filters) for the aseptic attachment by the end user of solutions, satellite bags, ancillary tubing and other sterile connecting devices for filters. The term open system, as applied to a leukapheresis tubing set, is used to define tubing sets which are only partially assembled at the device manufacturing site and are then customized and sterilized by the end user immediately prior to use.

Apparatus for carrying out centrifugation leukapheresis is described in U.S. Pat. No. 3,489,145 and U.S. Pat. No. 3,655,123, while that for carrying out filtration leukapheresis is described in U.S. Pat. No. 3,802,432 and U.S. Pat. No. 3,892,236. Gravity leukapheresis, in which the forces for both separating and collecting leukocytes are provided by gravity alone, is described in U.S. Pat. No. 4,111,199. Examples of automated leukapheresis apparatus now commercially available include the Fenwal CS-3000 (Baxter Healthcare, Chicago, Ill.), the Cobe 2997 (Cobe BCT, Lakewood, Colo.), the Cobe Spectra, the Cobe 2991, and the Haemonetics V50 (Haemonetics Corp., Braintree, Mass.).

### Use of Apheresis in Cell Banking

Cell banking is a service industry in which live cells are stored for later use. It has been practised for decades, and is exemplified by the storage of bovine sperm cells for the artificial insemination of cows.

With the technical advances that are being made in bio-medical research and tissue engineering, it is being recognized that many possibilities may exist for the use of human stem cells for various replacement therapies. These developments have led to a growing demand for facilities where stem cells of individuals can be isolated, cryo-preserved, and stored for later (autologous) use. For example, the desirability of storing the cord blood stem cells of newborns is becoming increasingly recognized and as a result there is a rapidly increasing number of deposits of such stem cells in private cell banks. Apheresis can be used to produce blood fractions enriched in stem cells from various hematopoietic tissue specimens, including bone marrow, peripheral blood (usually after stem cells have been mobilized from bone marrow by pre-administration of various growth factors) and cord blood. Apheresis can also be used to obtain many other types of cell, including T-lymphocytes and platelets.

With this growth in interest in cell and tissue banking has come an increasing awareness of the practical problems. It has become clear that cell banks intended to provide a long-term cellular resource are vulnerable to random events that lead to loss of viability of some or all of the deposits and that the risks associated with such events increase with the size of the bank and with the duration of storage. Deposit integrity is also crucially important: the way in which the deposits are prepared, stored, handled and used may crucially determine the integrity of the bank. This is particularly important when cross-contamination of deposits can lead to the spread of disease or to inappropriate or dangerous physiological consequences (such as may arise from the administration of allogenous cellular material when autologous grafting is indicated). With large banks, information storage, processing and deposit cataloguing are also extremely important.

Such issues have lead to a growing number of statutory provisions and codes of practice governing the production, maintenance and use of cell banks in most countries: in the United Kingdom, cell banking is now controlled by a comprehensive regulatory framework.

### Use of Apheresis in Contingent Autologous Transplantation (CAT) Therapy

A form of therapy has recently been described (see WO 00/29551 and WO 01/88099) in which various tissues (including leukocytes) are removed from a healthy donor and stored in a tissue or cell bank for later autologous transplantation in the event that a need for such autotransplantation arises at some future date. This form of therapy is herein referred to as contingent autologous transplantation (CAT) therapy.

For any given tissue or cell type, the need for CAT therapy is likely to arise in only a fraction of the healthy population. As a result, the effectiveness of CAT therapy depends crucially on the generation of comprehensive cell and tissue banks in which deposits from a large percentage of the population are included.

Accordingly, it has been proposed that CAT therapy be facilitated by the construction of comprehensive tissue banks. However, the nature of CAT therapy places unique and stringent demands on any such tissue bank. In particular, CAT therapy implies a large number of participating donors (and consequently a large number of deposits), relatively long-term storage, good retention of tissue function over time and great flexibility in ultimate therapeutic use.

Such problems are particularly acute in the case of leukocyte cell banks, where the absolute number of cells available is relatively small, the ultimate therapeutic efficacy may depend critically on the function of a small subset of cells and the activity profile of the stored leukocytes may change over time as the various subsets of cells respond to storage in different ways. To date, no leukocyte cell banks suitable for CAT have been constructed.

The present inventor has now recognized that the use of apheresis in the production of cell banks is greatly facilitated by the use of apparatus (and in particular apheresis tube sets) which permit cell harvesting, processing, collection, cryopreservation and banking to be completed with a closed (or functionally closed) system, so avoiding the need for expensive positive-pressure sterile room facilities and attendant specially trained staff.

Moreover, contingent autologous transplantation of leukocytes requires specific combinations of blood processing techniques and cell bank construction is necessary in order to meet the unique demands imposed on a leukocyte cell bank by CAT therapy, which include inter alia the need for reliable matching of autologous material, exceptionally robust long-term storage, retention of leukocyte functionality and flexibility in ultimate therapeutic potential.

In particular, the present inventor has found that specially-adapted leukapheresis tubing sets are necessary if blood is to be processed by the use of automated leukapheresis apparatus in order to fulfil the unique demands made on a leukocyte bank intended to support CAT therapy or to permit apheresis-based cell harvesting, processing, collection, cryopreservation and banking to be completed with a closed (or functionally closed) system.

### SUMMARY OF THE INVENTION

In a first aspect the invention provides a closed (or functionally closed) apheresis tubing set comprising a cell collect bag having two or more independent cell storage compartments.

The collect bag is preferably a cryocyte bag (e.g. a cryogenic leukocyte collect bag).

The tubing set is preferably manufactured out of PTFE (e.g. Teflon®).

As used herein, the term closed, in the context of the packs of the invention, is used to define blood packs consisting of elements which are sterile and isolated from the outside environment by aseptic barrier(s) and in which all components are fully integral, being attached and/or assembled at the manufacturing site.

As used herein the term functionally closed, in the context of the packs of the invention, is used to define blood packs consisting of elements (e.g. tubing sets) which are assembled at the device manufacturing site and which use sterile barrier filters (e.g. 0.22 micron filters) or so-called “docking systems” for the sterile interconnection by the end user to generate a wide variety of arrays of tubing, channels, filters, satellite bags and other vessels.

The term independent, as applied to the leukocyte storage compartments of the invention, is intended to define compartments which can be separated and independently stored (as defined herein). Thus, the independent storage compartments of the invention may have non-contiguous internal volumes, may not share a barrier and/or may constitute entirely separate (or separable) vessels.

The term independently stored defines a condition in which the leukocyte storage compartments are stored without sharing a determinant of viability selected from:

(a) power supply and/or (b) site or location. For example, in cases where the storage systems employed depend on a supply of electricity for their continued cryopreservation, then the electricity supplies must not originate from a single generator or supplier.

Preferably, each independent storage system is sited to be geographically remote from its counterpart(s), so lessening the chances of coincidental destruction or damage by natural or man-made disasters (such as fire, flood or contamination).

Preferably, the bag has three storage compartments. The provision of two or more (preferably three) storage compartments facilitates the construction of cell banks which exhibit deposit redundancy (see infra).

The storage compartments are preferably releasably joined. This facilitates loading and handling of the tubing set during the blood processing stage whilst permitting ready separation of the compartments after cell collection for subsequent independent storage.

Any suitable form of releasable join may be employed, and particularly convenient is the provision of one or more breakable bridges (for example, a perforated or scored connecting strip). Since the bag is conveniently formed from two leaves of a flexible, semi-rigid and/or transparent plastics material (e.g. PTFE), the storage compartments may be defined by heat seams and the connecting strip may take the form of a perforated heat seam.

The bag preferably comprises bipolar suspension means. The term bipolar is used here to define suspension means which are located at opposite ends of the bag, so permitting it to be hung in two orientations, one orientation being 180° rotated relative to the other. As described infra, this facilitates the staged feeding of cryoprotectant and leukocyte material into the bag. The suspension means may take any form, but preferably takes the form of one or more holes.

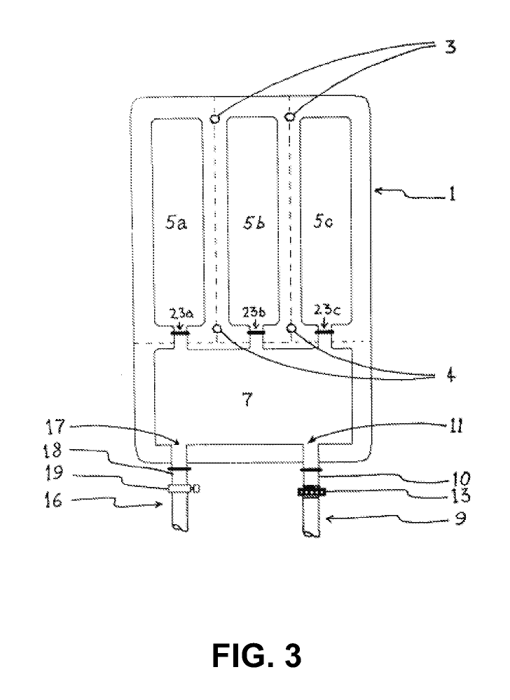

Each of the storage compartments may be provided with a cryoprotectant port and a cell sample port. In some embodiments, the sample and cryoprotectant ports are located at opposite sides of the bag. In other embodiments (e.g. described hereinafter and illustrated in FIG. 3) sample and cryoprotectant ports are located on the same side of the bag.

The cryoprotectant port is preferably connected to a cryoprotectant inlet by a conduit. The cryoprotectant inlet may take any form, so long as it permits the introduction of cryoprotectant into the storage compartment via the port. The inlet may therefore comprise a sterile docking port, valve, luer lock, breakable seal or blind sack (which can be pierced with a spike and then filled with cryoprotectant).

In preferred embodiments, the cryoprotectant port can be sealed, for example by clamping of the conduit.

The conduit may also take any form provided that it provides for fluid communication between the cryoprotectant inlet and cryoprotectant port. Preferably, the conduit takes the form of a flexible or semi-rigid tube, e.g. of transparent plastics material (e.g. PVC or PTFE).

The conduit may take the form of a tail attached to the bag in the region of the cryoprotectant port. As used herein, the term “tail” is used to define a section of conduit which is fixed to the bag in a separate operation. The conduit tail is usually, but not necessarily, of a different material to the bag (typically, the bag is of fluorocarbon polymer while the conduit tail is of PVC).

The cryoprotectant port is preferably separated from the cryoprotectant inlet by a sterile barrier filter (e.g. a 0.22 micron filter) located in the conduit downstream of the inlet. This permits cryoprotectant to be introduced at the inlet from an open (non-sterile) source so that incoming cryoprotectant enters a closed system and is sterilized after passing through the filter.

The conduit preferably branches into the cryoprotectant port of each of the storage compartments at a manifold. In such configurations, a single sterile barrier filter is preferably located between the cryoprotectant inlet and the manifold so that each branch of the incoming cryoprotectant line is closed by a single filter.

In such configurations the cryoprotectant may be pumped (e.g. manually with a syringe) through the manifold into each storage compartment and if this is performed against the action of gravity then the volume of cryoprotectant passing into the manifold and the storage compartments may be more easily controlled and the introduction of equal aliquots of cryoprotectant into each storage compartment facilitated.

The cell sample (e.g. leukocyte) port is preferably connected to a leukocyte inlet by a conduit. Again, this conduit may also take the form of a tail attached to the bag in the region of the cryoprotectant port, so that the cell sample conduit tail may be of a different material to the bag (typically, the bag is of fluorocarbon polymer such as Teflon®, while the sample conduit tail is of PVC).

The use of such tails may facilitate the use of sterile docking apparatus (such as the Terumo® sterile tube welding apparatus or other similar machines, as described in e.g. EP0507321) to effect interchange between different apheresis tube sets and cryocyte bags whilst maintaining a functionally closed system. It also facilitates the use of plastics materials optimised for prolonged cryopreservation (such as fluorocarbon polymers) in the cryocyte bag in combination with plastics optimised for use at room temperature within an automated apheresis machine for the rest of the tubing set. For example, PVC is commonly used to manufacture apheresis tubing sets, but this plastic is not ideal as a cryocyte bag material (it becomes brittle at very low temperatures). The use of PVC-tailed Teflon® cryocyte bags permits such bags (which are far more robust at low temperatures) to be used as interchangeable modules with a wide range of different PVC apheresis tubing sets within a functionally closed system simply by using a suitable sterile tube welding apparatus to attach the bag to the tubing set.

The cell sample (e.g. leukocyte) inlet may take any form, so long as it permits the introduction of separated cells into the storage compartment via the cell sample port. The inlet may therefore comprise a sterile docking port, valve, luer lock, breakable seal or blind sack (which can be pierced with a spike and then filled with sample).

In preferred embodiments, the conduit preferably branches into the leukocyte port of each of the storage compartments at a manifold. The leukocyte ports may be sealed, for example by clamping of the conduit.

In such configurations the leukocytes may be pumped (e.g. manually with a syringe) through the manifold into each storage compartment and if this is performed against the action of gravity then the volume of leukocyte sample passing into the manifold and the storage compartments may be more easily controlled and the introduction of equal aliquots into each storage compartment facilitated.

The tubing set preferably further comprises a blood (or bone marrow) processing vessel. Where present, the blood/marrow processing vessel may comprise: (a) a centrifuge loop; or (b) a Latham bowl; or (c) a filtration device.

The blood/marrow sample for use in the process of the invention may be one which is in fluid communication with the donor individual (for example, in circumstances where leukapheresis is used to selectively separate the leukocytes). Preferably, however, the sample is an isolated blood/marrow sample (as defined herein). Where cord blood is used as a source of cells, the blood sample is of course necessarily isolated. In such cases, the tubing set may also further comprise a sample vessel for holding an isolated blood/marrow sample. It may also further comprise means (e.g. comprising a needle) for collecting an isolated blood sample from an individual.

The apheresis tubing set of the invention may be specifically adapted for use in an automated apheresis device, preferably a continuous or interrupted flow centrifugation apheresis or continuous or interrupted flow filtration apheresis device.

Any of a wide variety of commercially available apheresis devices may be used according to the present invention. The particular way in which the device is operated will depend on a number of factors, including the nature of the separation device (e.g. centrifuge, filter etc.), the type of sample required, the volume of the blood/marrow sample to be processed, the identity and status of the donor individual, the ultimate use to which the cell composition is to be put and the nature of any treatments applied to the sample prior to processing according to the invention. Thus, those skilled in the art will readily be able to establish the appropriate operational parameters.

Preferably, however, the apheresis device is selected to minimize the need for operator intervention and/or training Commercially available apheresis systems vary in the time and/or expertise required of an individual to prepare and operate it. For instance, reducing the time required by the operator to load and unload the tube set, as well as the complexity of these actions, can increase productivity and/or reduce the potential for operator error. Moreover, reducing the dependency of the system on the operator may lead to reductions in operator errors and/or to reductions in the credentials desired/required for the operators of these systems.

Performance-related factors are also relevant, and may be judged inter alia in terms of the “collection efficiency” of the apheresis system. The “collection efficiency” of a system may of course be gauged in a variety of ways, such as by the size of the fraction of cells (e.g. leukocytes) collected in relation to the total cells present in the sample. Performance may also be evaluated based upon the effect which the apheresis procedure has on the various blood component types. For instance, it may be desirable to minimize the adverse effects on at least the leukocytes of the apheresis procedure. It may also be desirable to reduce platelet activation, in order to avoid degeneration in sample quality during processing.

Particularly preferred is the Cobe® system (Cobe BCT, Lakewood, Colo., USA). In such embodiments, the apheresis tubing set of the invention may comprise the COBE Spectra™ Automated Peripheral Blood Stem Cell Set (AutoPBSC Set), with the “collect bag” replaced with the cryocyte bag of the invention.

Thus, in another aspect the invention contemplates apparatus for selectively separating and removing cells from an isolated blood/marrow sample comprising an apheresis device loaded with the tubing set of the invention.

The apparatus of the invention preferably comprises the tubing set of the invention together with a separation device (e.g. a centrifuge rotor or filter) and one or more pumps for conveying the blood sample through the tubing set.

In another aspect, the invention contemplated a cryocyte or leukocyte collect bag for use with the tubing set as defined in any one of the preceding claims.

In another aspect, the invention provides a process for producing a leukocyte composition suitable for CAT therapy comprising the step of selectively separating and collecting leukocytes from a donor using the leukapheresis device of the invention.

Any donor may be used as a source of blood sample in the processes of the invention, provided that the donor is healthy, as herein defined. However, the invention finds particular application in relation to donor individuals which are predisposed to a leukocyte deficiency, are not in remission from a leukocyte deficiency, are juvenile, adolescent or adult, are at risk of developing a leukocyte deficiency, are human individuals between the ages of about 12 to 30 (e.g. 15 to 25) and/or have a fully-developed immune system. However, in some applications where the donor individual is an adult donor individual, then the donor's age may be at least 30, 40, 50, 60 or 70 years.

The invention also contemplates a cell composition and a cell bank obtainable (or obtained) by the process of the invention.

Also contemplated are various therapeutic uses for the processes, compositions and banks of the invention. Accordingly, the invention contemplates the leukocyte composition of the invention for use in therapy, for example in CAT therapy and in other forms of autotransplantation (e.g. in restorative or remedial autotransplantation).

In another aspect, the invention provides a process for producing a cell bank (e.g. a leukocyte cell bank suitable for CAT therapy) comprising the steps of:
(a) selectively separating and collecting cells (e.g. leukocytes) from the sample using the apheresis device of the invention;
(b) cryogenically preserving the cells (e.g. leukocytes); and
(c) applying steps (a) and (b) iteratively to a series of blood samples from different healthy donor individuals.

The process may also comprise retrievably depositing the preserved cells (e.g. leukocytes) into two or more independent storage systems to produce a bank which exhibits deposit redundancy. As a further precaution, two or more aliquots are used according to the invention in order to provide for deposit redundancy. Preferably, three, four, five or greater than five separate aliquots are used according to the invention.

The independent storage systems into which the aliquots are retrievably deposited are independent in the sense that they do not share a determinant of viability selected from: (a) power supply and/or (b) site or location. For example, in cases where the storage systems depend on a supply of electricity for their continued cryopreservation, then the electricity supplies must not originate from a single generator or supplier.

Preferably, each independent storage system is sited to be geographically remote from its counterpart(s), so lessening the chances of coincidental destruction or damage by natural or man-made disasters (such as fire, flood or contamination).

The separation and collection steps are preferably conducted within a closed or functionally closed system and may be applied iteratively to a series of blood samples from different healthy donor individuals.

The process may further comprise the step of digitally storing information obtained from each donor individual in a digital information unit so as to permit matching of deposit and donor for later autologous transplantation.

The information stored comprises at least that necessary to permit matching of deposit with donor, in order that later autologous transplantation can be carried out. Preferably, the information comprises genetic information, the date at which the blood sample was collected from the donor individual, the age and sex of the donor individual, the clinical status of the donor individual, the medical history of the donor individual, biographical data identifying the donor individual, details of the processing and storage conditions used to prepare the deposit as well as data identifying the person(s) responsible for processing the sample(s).

If genetic information is stored, then this preferably comprises sequence information relating to one or more gene(s), single nucleotide polymorphism (SNP) data and/or one or more genetic fingerprint(s).

Any suitable digital information unit may be used to store the information. Preferably, this takes the form of at least one digital computer comprising a database. The database may carry data on a carrier of any convenient form. Preferably, the information is stored independently on two or more carriers so that the database exhibits redundancy. This protects against data loss in the event of failure, corruption or loss of one of the computers or data carriers.

Preferably, the process further comprises the step of labelling the storage vessels with information sufficient to permit matching of the leukocyte deposit and donor. For example, the storage vessels may be labelled with information:
(a) describing the contents of the vessel (for example, sample size, number and/or volume); and/or
(b) identifying the leukocyte bank; and/or
(c) recording the date at which the blood sample was collected from the donor individual; and/or
(d) comprising a statement that each package is for single patient use only; and/or
(e) comprising instructions for opening, aseptic presentation and further storage.

Any convenient form of labelling may be used. Thus, the labelling may comprise the physical attachment of an information carrier (e.g. a bar code) to the storage vessels themselves. Alternatively, the labelling may be effected by the non-physical association of the vessels with the information carrier (for example, via the correlation between the physical geometry or organization of the deposits in the bank and the entries in the database). In preferred embodiments, a freeze-resistant radiofrequency identification device (RF ID) is used. Such devices are commercially available and greatly facilitate accurate and rapid sample identification. Such devices may also comprise thermocouples, so facilitating the recovery of data describing any temperature fluctuations to which the sample was exposed during storage.

The invention also contemplates treatment of the leukocytes, for example including any or all of the following: in vivo prior to provision of the blood sample, in vitro prior to separation step (b), in vitro after separation step (b) but prior to preservation step (f) and/or in vitro after preservation step (f).

The invention also contemplates a leukocyte composition and a leukocyte cell bank obtainable (or obtained) by the process of the invention.

Also contemplated are various therapeutic uses for the processes, compositions and banks of the invention. Accordingly, the invention contemplates the leukocyte composition of the invention for use in therapy, for example in autotransplantation (e.g. in restorative or remedial autotransplantation).

### DETAILED DESCRIPTION OF THE INVENTION

### I. Definitions

Where used herein and unless specifically indicated otherwise, the following terms are intended to have the following meanings in addition to any broader (or narrower) meanings the terms might enjoy in the art:

The term leukapheresis is a term of art used herein to define a procedure involving the selective separation and removal of leukocytes from the withdrawn blood of a donor, the remainder of the blood then being retransfused into the donor.

A leukapheresis device, apparatus or machine is a term of art defining any device capable of performing leukapheresis, irrespective of the means employed in the device to separate and remove the leukocytes.

The term tubing set is a term of art defining a blood processing vessel and an array of channels (usually tubes) which together hold the blood and its various fractions in a particular geometry when used in conjunction with a leukapheresis device.

The term cell collect bag defines that portion of a tubing set within which cells (e.g. leukocytes or other blood or bone marrow fractions) are collected (and optionally stored). The term leukocyte collect bag defines that portion of a tubing set within which leukocytes (or other blood or bone marrow fractions) are collected (and optionally stored).

The term cryogenic leukocyte collect bag is a term of art used to define a container designed for holding leukocyte samples under sterile conditions during long-term (e.g. at least one year) cryopreservation at temperatures of less than −160° C.

The term cryocyte bag is a term of art used to define a container designed for holding cell samples under sterile conditions during long-term (e.g. at least one year) cryopreservation at temperatures of less than −160° C. Exemplary cryocyte bags include the cryogenic cell (e.g. leukocyte) collect bags as defined above.

The term patency tube or patency tubing is used herein to define tubing the lumen of which is maintained in an open (patent) state by the structural properties of the tubing under circumstances where the inner walls of the tube are wetted (e.g. with a lymphocyte sample). Such tubing need not be circular in cross section: any tubing geometry may be employed provided that the walls of the tube maintain an open lumen when the inner surfaces of the tube are wetted. When disposed between two compartments in a cryocyte bag, patency tubing facilitates fluid communication between the compartments. When incorporated into the sample retrieval port of a cryocyte bag, patency tubing can facilitate sample retrieval after storage and thawing by maintaining an open retrieval port and preventing surface tension forces from occluding it.

The term blood processing vessel defines that portion of the tubing set within which the leukocytes are subjected to separation forces in the separation device.

The term isolated leukapheresis is used herein to define a novel form of leukapheresis which is performed on an isolated blood sample.

The term isolated blood sample is used herein to define a blood sample which is not in fluid communication with the blood of the donor from which it originated. Thus, in the process of isolated leukapheresis which is applied to isolated blood samples, the leukapheresis device is not in fluid communication with the individual providing the blood sample and/or the remainder of the blood in the sample is not retransfused into the individual. Cord blood samples are necessarily isolated in practice. In some applications, the use of isolated peripheral blood (or bone marrow) samples may be preferred.

The term autotransplantation is used herein to define autologous transplantation (autogeneic or self-to-self transplantation), wherein the term autologous is used to indicate that the transplantation is to the same organism (i.e. the same individual) from which the cellular material (e.g. leukocytes) was removed. As used herein, transplantation defines any procedure involving the introduction of cellular material (e.g. leukocytes) into an organism, and so any form of transplantation or grafting known in the art is encompassed.

The term dormancy is used herein to define any state of suspended animation or stasis, and procedures for achieving this are well known in the art, as described below. Any of the known procedures may be used, including cryopreservation. Thus, the leukocytes may be held or maintained in a quiescent, inactive or non-proliferating state.

The term healthy is used herein in relation to an individual donor to indicate that the individual is not suffering from a leukocytic deficiency (as herein defined). Thus, the term healthy as used herein encompasses non-diseased individual donors in a state in which the individual donor is not suffering from any disease or disorder, or is not manifesting any symptoms of said disease or disorder (i.e. is asymptomatic or is in a pre-clinical condition). In particular, term healthy as used herein encompasses individual donors not suffering from, or demonstrating symptoms of, the disease or disorder which it is subsequently intended to treat by the autotransplantation procedure.

### II. Cell Sources

The tubing set of the invention finds application in the processing (e.g. collection and storage) of any kind of cellular material, and in particular in the processing of any form of hematopoietic tissue, including blood or bone marrow samples. For example, the blood sample may comprise cord blood or peripheral blood. Preferably, the tubing set is used in the processing and storage of leukocytes, when the blood sample may be an isolated blood sample, as defined herein.

The blood sample may be subjected to various treatments ex vivo prior to use in the process of the invention. Typically, for example, the blood sample is chilled prior to use. Other treatments may include the addition of preservatives and/or anticoagulants.

The blood sample may also be treated in vivo prior to collection by administering various agents to the donor individual before or during sample collection.

Examples of treatments (which may be applied ex vivo and/or in vivo) are discussed in more detail in the section entitled “Leukocyte treatments”, below.

It is generally preferable to sample at least 450-500 ml of blood from the individual donor, which is the equivalent of a unit of blood as provided by a blood donor for the UK blood transfusion service. If possible a number of samples (e.g. several 450-500 ml samples) are taken over a period of time (e.g. over 2-3 weeks, preferably 2-3 months or over 6 months or a year, 2 or 3 years or more). One or more of these can then be divided or combined into a number of leukocyte cell bank deposits. The removal of a unit of blood is commonplace with over three million units of blood being taken, for allografting, from individuals annually in the UK alone.

The blood removed is soon replaced and, therefore, multiple samplings of a unit of blood from an individual can be provided over a year, say 2-12 unit samplings if necessary, without detriment to the individual being sampled.

The invention therefore finds utility in the processing of cellular material from inter alia bone marrow and cord blood (including for example peripheral mononuclear cells).

### III. Selection of Donor Individuals for Cat Therapy

### General Considerations

Restorative autotransplantation is a form of therapy that might ultimately be indicated for any individual. Consequently, the invention may be usefully applied to the generation of comprehensive leukocyte cell banks covering as large a number of different individuals as possible in order that restorative autotransplantation can be carried out in any of the represented individuals should the need arise.

It is therefore contemplated that the invention be applied as broadly as possible so that a comprehensive leukocyte cell bank can be assembled. However, since the quality of the individual deposits will depend (at least to some extent) on the health status of the individual donor at the time of blood sample donation, it is preferred that the blood sample for use in the processes of the invention be taken from healthy individual donors.

Other factors also affect donor selection: for example, the blood sample for use in the processes of the invention may advantageously be obtained from individual donors when they are young, preferably in adolescence or early adulthood. In the case of humans, blood sampling (preferably multiple sampling) at the ages of about 12 to 30, preferably 15 to 25 is preferred. Especially preferably, sampling is from the age of 16 or 17 upwards, for example in the age range 16 to 30, 17 to 30, or 18 to 30, or perhaps 18 to 35 or 40. It is thus preferred that the cells be obtained when the host organism is mature, or reaching maturity, but before the processes of ageing or senescence have significantly set in. In particular, it is preferred and advantageous that the immune system of the host organism is mature or fully developed.

However, the obtention of cells outside these ranges is encompassed, and cells may be obtained at any post-natal life stage e.g. from juvenile host organisms e.g. in mid-to late childhood, or even infants, or from older individuals.

Sampling from post-natal or older hosts allows multiple samples to be collected, thereby increasing the opportunity of storing sufficient number of cells. In addition sampling from juvenile or older hosts overcomes the ethical requirements such as providing informed consent.

Sampling from adolescent or adult host organisms is preferred since the sampled cells, from blood in particular, will contain a greater proportion of valuable mature T-cells capable of recognising aberrant cell populations, such as cancer cells or virally infected cells. Thus, when blood samples are used, it is advantageous that they are taken from an individual with a mature immune system (i.e. not foetal or neonatal).

Thus, the invention contemplates the use of blood samples collected from donor individuals at a stage when there is no direct prediction, suggestion, or suspicion that a particular disorder or disease may develop, for use against a future possible or unpredicted event, or an event which may occur simply by chance, rather than an anticipated or suspected or predicted illness or condition. Thus, in certain embodiments of the invention, the donor individual is not predisposed to, or at risk from, any particular disease or disorder e.g. not exhibiting any symptoms or manifestations predictive of a subsequent disease or disorder. Likewise, the host organism is preferably not suffering from any injuries or damage which may give rise to an anticipated or expected condition.

Indeed, for certain applications (for example, the generation of leukocyte cell banks for subsequent restorative autotransplantation) it is preferred that the blood sample for use in the invention be obtained from the donor individual before any disease or disorder develops or manifests itself, and more preferably when the host organism is in general good health, and preferably not immunocompromised in any way. In such embodiments it is particularly advantageous to sample the blood from donor individuals at a time when the organism has not previously exhibited symptoms of or presented with or been diagnosed as suffering from the disease or disorder which is subsequently to be treated, i.e. when the host organism is healthy and not “in remission” e.g. not in a state of partial or full recovery from the leukocyte deficiency to be treated.

### Predisposed Donor Individuals

Advances in therapy continue to be made, and our greater understanding of disease processes helps us to modify and refocus our therapeutic approaches to alleviate disease and suffering. Such understanding has been greatly advanced by technological improvements in the field of molecular biology. We are now in a position to follow the pathogenesis of diseases at a molecular level, and recognize the importance of an individual's genetic make-up in predisposing them to certain diseases. For example, we are aware that some individuals, because of their genetic composition, are prone to certain diseases.

Many of the diseases to which certain individuals can be predisposed are leukocyte deficiencies, which term is used herein to indicate a condition in which the administration of autologous leukocytes is indicated. Such conditions therefore include those in which an individual has acquired a disease, infection or condition involving leukocyte dysfunction or a disease, infection or condition in which the augmentation or stimulation of endogenous leukocyte activity is indicated. Detailed examples of particular leukocyte deficiencies are set out in the section entitled “Exemplary indications”, below.

Through genetic testing, therefore, it is now possible to identify those individuals predisposed to a leukocyte deficiency (e.g. any of various forms of cancer, immune disorder or infection).

Furthermore, our knowledge of the body's immune system, and in particular the way in which it recognises and kills virally infected and tumour cells, continues to advance. We now know that in order to elicit cell-mediated immunity, an offending cell (e.g. a virally infected or tumour cell) must co-present an HLA class I restricted tumour or viral epitope with danger signals such as GM-CSF and/or TNF-alpha, so that the antigen presenting cells (APC) of the immune system will express co-stimulatory signals such as B7 and IL-12 in conjunction with antigen to the interacting cytotoxic T-lymphocyte (CTL) population. The co-presentation leads to the production of clones of both activated and memory cells (for review see Nature Medicine Vaccine Supplement 4 (1998) 525). In the absence of these additional signals, HLA-I antigen-restricted T-cells which recognise offending cells are processed for destruction or desensitization (a bodily process presumably put into place to avoid the development of e.g. autoimmune disease). The induction of such tolerance is because of either ignorance, anergy or physical deletion (Cold Spring Harbour Symp Quant Biol 2 (1989) 807; Nature 342 (1989) 564; Cell 65 (1991) 305; Nature Med 4 (1998) 525).

It is now clear that tumour cells do not automatically co-present danger and/or co-stimulatory signals. Hence, the spawning of a tumour may lead to eradication of the very T cell clones that provide cell-mediated immunity against the tumour. A patient presenting with a cancer, leukaemia/lymphoma or sarcoma etc, therefore, may have already removed their innate ability to destroy the tumour, by default.

However, if the required T lymphocytes, or a sample thereof, were removed from the patient prior to the onset of proliferative disease, the relevant T-cell population could now be returned to the patient, after any necessary co-stimulation of the T-cells, so as to alleviate disease. Co-stimulation may be provided at the same time as the cells are returned to the patient, or after they are returned through further treatment (s) of the patient, or without stimulation other than that naturally produced by the patient. Activation/stimulation of the cells may also initially be induced in vitro prior to reinfusion.

The present invention therefore finds particular application in the case of individuals predisposed to the development of a leukocyte deficiency. It therefore represents a means for removing leukocytes from a healthy donor individual for subsequent transplantation to that same individual in a subsequent autologous (autogeneic) transplantation procedure, when the need or desire to do so arises. Although the predisposed individual may never receive the cells because no disease to be treated by this method ever occurs, the invention nevertheless may be used to provide some form of insurance against the heightened risk of a leukocyte deficiency arising in the individual.

Similarly, individuals with no diagnosed predisposition may choose to provide samples for incorporation into the leukocyte cell bank of the invention for prospective use by themselves prior to travelling abroad. Such use might include for the treatment of infections contracted whilst abroad.

In addition, it is well recognized that the ageing process makes individuals more susceptible to disease. The basis for the susceptibility appears to be in the loss of immune function resulting from a significant decrease in T and B cell numbers/activity during ageing (Mech Ageing & Dev 91 (1996) 219; Science 273 (1996) 70; Mech Ageing & Dev 96 (1997) 1). Disease susceptibility is particularly pertinent when elderly patients are subjected to e.g. surgery in a hospital environment, where they are prone to opportunistic infections with serious or even fatal consequences. Blood samples taken from such individuals much earlier in life and processed according to the invention for inclusion in a leukocyte cell bank could provide the opportunity for restorative autotransplantation in such circumstances.

Such an approach could be used more broadly to provide for a method of augmenting the patient's immune system after surgery in order to lessen the likelihood of post-operative complications caused by opportunistic infections. The invention, therefore, could be used as a prophylactic therapy, e.g. for elderly patients when they are more susceptible to disease.

### IV. Leukocytes

It will be appreciated that the separation and/or removal of leukocytes from the blood sample during such processing need not be absolute. Rather, the removal and/or separation of a fraction of the total leukocytes present in the sample is sufficient in most circumstances. Those skilled in the art will readily be able to determine the appropriate size of the fraction to be removed, which will vary inter alia according to the use to which the isolated leukocytes are to be put, the size of the sample, the status of the donor and the nature of the leukocytes.

The leukocytes collected in the processes of the invention are to some degree isolated from the original blood sample. The term isolated is used here to indicate that the isolated leukocytes exist in a physical milieu distinct from that in which they occur in vivo and does not imply any particular degree of purity. Indeed, the absolute level of purity is not critical, and those skilled in the art can readily determine appropriate levels of purity according to the use to which the leukocytes are to be put.

The separation and collection of the leukocytes in the processes of the invention also does not necessarily imply that any particular class or type of leukocyte is preferentially separated and collected. Rather, the leukocytes of the invention include any white blood cell, including granulocytes, lymphocytes and monocytes.

Granulocytes include myelocytes, basophils, eosinophils and neutrophils. Lymphocytes include B, T lymphocytes and natural killer cells. Monocytes include mononuclear phagocytes and other macrophages.

However, in some embodiments the leukocytes which are separated and collected preferably comprise one or more specific leukocyte cell types. A preferred cell type is the lymphocyte, especially a T-lymphocyte (T-cell). Mature T-lymphocytes are particularly preferred.

Since the total mature T-cell number per litre of blood ranges between 1-2.5×109 for humans, a 100 ml sample of blood typically contains 1-2.5×108 mature T-cells and this is generally sufficient to provide an adequate representation of the entire mature human T-cell population for the beneficial effect. However, depending on the fraction of total leukocytes separated and collected and the efficiency of any revitalizing technique employed, preferably at least 100 ml, 115 ml, 200 ml or 300 ml and even more preferably in excess of 400 or 500 ml of blood sample is used in order to obtain the appropriate number of mature T-cells to support a beneficial therapeutic effect for return to the individual if and when they become ill.

Standard techniques are known in the art which permit selection of particular subpopulations of lymphocytes from a sample comprising a mixed population of lymphocytes. Examples of such subpopulations are CD3+, CD8+, CD4+ and CD16/56+ (natural killer) T cells and CD19+ B cells. For example, any one or any mixture or combination of such subpopulations of T cells can be used in the methods, uses and compositions of the invention, and they are readily obtained by means of well known methods such as FACS (Fluorescence Activated Cell Sorting) and haemocytometry systems.

### V. Leukocyte Treatments

The leukocytes may be subjected to various treatments. Such treatments may, for example, result in expansion of some or all of the representative cell subsets, improve the long-term viability of the leukocytes during the dormancy period, improve their therapeutic potency, remedy a deficiency or defect exhibited by some or all of the leukocytes (as is the case, for example, in remedial autotransplantation therapeutic modalities) and/or render their subsequent use in autotransplantation safer.

The treatments can be carried out before or after the leukocytes are rendered dormant (and before or after autotransplantation is carried out). Moreover, the treatments may be applied after the blood sample is taken (i.e. be carried out ex vivo) either prior to rendering the cells dormant or after revitalization. For example, treatment of the leukocytes may be effected by co administration of a separate composition, sequentially or simultaneously with the leukocyte composition, during autotransplantation. Treatment of the leukocytes can be effected immediately prior to autotransplantation.

Alternatively (or in addition) the treatments may be applied to the leukocytes while still in vivo prior to blood sampling by the administration of e.g. growth factors or cytokines (see below).

Exemplary pre-transplantation treatments may include various genetic modifications, such as the incorporation of a negative selection marker (as described, for example, in WO96/14401, the content of which is incorporated herein by reference). Such treatment permits ablation of the leukocytes after transplantation or titration of dose versus response. Other genetic interventions may include regulating or modifying the expression of one or more genes (e.g. increasing or decreasing gene expression), inactivating one or more genes, gene replacement and/or the expression of one or more heterologous genes). Other genetic modifications include the targeting of particular T-cells (as described in WO96/15238, the content of which is incorporated herein by reference), and the modification of the T-cell receptor repertoire/expression with antibodies to make T-cell chimaeras.

Other treatments contemplated by the invention include the exposure of the leukocytes with one or more stimulatory molecules, for example antigens (e.g. cancer or viral antigens), antibodies, T cell recognition epitopes, peptides, blood factors, hormones, growth factors or cytokines or combinations thereof.

For example, the leukocytes may be treated in vitro (or in vivo prior to blood sampling) with antigens (for example cancer (e.g. prostate-specific antigen 1 or prostate-specific antigen 2, her-2/new, MAGE-1, p53, Ha-ras and c-myc) or viral antigens), antibodies, T cell recognition epitopes, peptides, blood factors, hormones, growth factors or cytokines or combinations thereof. The stimulatory molecules may be synthetic, recombinant or may be purified or isolated from the human or animal body. Particularly useful in this respect are stimulatory molecules selected from IFN-alpha, IFN-beta, IFN-gamma, Il-1a, Il-1b, Il-2, Il-3, Il-4, Il-5, Il-6, Il-7, Il-8, Il-9, Il-10, Il-11, Il-12, Il-13, Il-14, II-15, GM-CSF, M-CSF, G-CSF, LT and combinations of two or more of the foregoing. Such treatments may modify the growth and/or activity and/or state of differentiation of the leukocytes, and/or may serve to separate or selectively isolate or enrich desired leukocyte cell types or to purge unwanted cells.

Recent advances have been made in the way cells may be obtained for subsequent autotransplantation. For example, investigations into the agents which regulate haematopoiesis have led to the isolation of a series of factors that influence the proliferation and differentiation of lymphocytes. These agents include the cytokines (such as the interleukin series IL-1 to IL-18 and the leukotrienes) and growth factors such as the TNF's, the TGF's, FGF's, EGF's, GM-CSF, G-CSF and others. A number of these factors are now available commercially for clinical use, and some have been shown to increase substantially the number of lymphocytic cells and, in particular, immature T-lymphocytes in the peripheral blood. Their administration to the donor individual prior to blood sampling permits the quantity and/or quality (in terms of the number and nature of leukocyte subtypes present) to be controlled and makes it possible to recover large quantities of the cells of interest, e.g. immature T-lymphocytes, directly from the donor individuals peripheral blood sample without the need to sample the marrow.

Other pre-transplantation treatments include culture of the leukocytes (or a sub-population thereof). For example, the leukocytes may be cultured to increase cell numbers. For example, the cells may be passaged, according to methods well known in the art. Such culturing may be carried out before or after the leukocytes are rendered dormant, or both before and after dormancy is induced.

Thus, in the case where the leukocytes include T-cells, the T-cells may be co-stimulated prior to transplantation and/or exposed to tumour antigens (optionally together with co-stimulatory factors) prior to autotransplantation.

The leukocyte treatments described above may be conveniently conducted within a separate culture compartment within the cryocyte bag. In this way, the leukocyte treatment(s) can be conducted entirely within a closed (or functionally closed) system. This aspect of the invention is described in more detail in Section XII, below.

### VI. Induction of Dormancy

Any suitable means may be employed for inducing dormancy.

According to a preferred cryopreservation procedure, the cells are frozen. The cryogenic preservation step conveniently comprises freezing to a temperature at or below about −160° C., which can be achieved using liquid nitrogen. If longer periods of storage and/or enhanced retention of functionality are required then freezing to a temperature at or below about −269° C. may be effected using liquid helium.

Any of a wide range of suitable cryopreservation media may be used according to the invention, but preferred are media comprising a suitable penetrating cryoprotectant. Particularly suitable for use as a penetrating cryoprotectant is DMSO, which may be used for example at a concentration of up to 10%.

The cryopreservation medium may further comprise an anticoagulant (such as acid citrate dextrose, EDTA, heparin or mixtures thereof), a nuclease (for example a Dnase and/or Rnase as well as a physiologically acceptable medium (for example, phosphate buffered saline). The cryopreservation medium may also further comprise a proteinaceous composition, such as blood serum, a blood serum component, blood plasma and/or a sugar and/or a polysaccharide (which may be particularly preferred in embodiments where plunge freezing is employed).

Particularly preferred proteinaceous compositions for use in the cryogenic preservation media of the invention comprise blood albumin (e.g. bovine serum albumin or human serum albumin). Particularly convenient is the use of human blood serum isolated from the blood sample of the donor individual. This can be isolated as a co-product together with the leukocytes.

As described in Freshney's (Freshney's Tissue Culture of Animal Cells (Culture of Animal Cells: A Manual of Basic Technique, Wiley Liss, 1994)), the cells may be suspended in a suitable medium (e.g. containing up to 10% DMSO) and cooled at a controlled rate (e.g. 1° C. per minute to −70° C., then into liquid/gas N2). Such conventional procedures may be adapted to cool the cells into He/N2 mixtures or He. Alternative methods of achieving and/or maintaining cell dormancy include cooling to 4° C.

### VII. Revitalization

Following dormancy, the cells are revitalised prior to use in transplantation. Again, this may be achieved in any convenient manner known in the art, and any method of revitalising or reviving the cells may be used.

Conveniently, this may, for example, be achieved by thawing and/or diluting the cells. Techniques for revitalisation are well known in the art. Cells may be thawed by gentle agitation of the container holding the cells in water at 37° C., followed by dilution of DMSO to 1% or below, e.g. with medium, plasma or serum.

Cells may be implanted immediately or after recovery in culture. Revitalisation is designed to re-establish the usefulness of the cells e.g. in prophylaxis or curative therapy.

### VIII. Cell Banking

The cell (e.g. leukocyte) compositions of the invention may be banked, thereby creating a cell (e.g. leukocyte) bank. Preferably, the compositions are banked after the cells have been rendered dormant (as described above).

Any suitable cell banking system may be employed, provided that the deposits are retrievable for autotransplantation. This implies the use of some form of labelling (e.g. etching, for example with a bar code), but this need not be in the form of a physical appendage to the individual deposits.

Thus, the cell bank of the invention may comprise a plurality of cell storage units for storage of cell compositions. Typically, such cell storage is effected by cryopreservation, but other storage techniques can also be employed. The cell banks of the invention may further include a digital information unit for digitally storing information relating to the identity, location and medical history of the donor individual and/or the conditions associated with the particular deposit (for example relating to the date at which the blood sample was collected from the donor individual, the processing conditions and details of any treatments applied to the leukocytes contained in the deposit).

The digital information unit preferably comprises at least one digital computer having sufficient digital storage capacity for storage of the potentially large amounts of information relating to each deposit.

The cell bank of the invention preferably further comprises an arrangement for digital data retrieval interfaced with the digital information unit for retrieving selected information stored in the digital information unit. The data retrieval arrangement may be integrated with the digital computer. Remote access of the digital information via the telephone or the internet may also be provided and may permit rapid and convenient access of the information on a global basis.

### IX. Medical Applications of Cryopreserved Leukocyte Compositions

The invention finds application in all forms of therapy and prophylaxis in which the administration of (treated or untreated) autologous leukocytes is indicated (i.e. desirable from a therapeutic perspective).

For the purposes of the present invention, in such indications a leukocyte deficiency is deemed to have arisen.

It will therefore be understood that the leukocyte deficiencies in which the invention finds medical application encompass a very broad spectrum of diseases, syndromes, disorders, conditions and infections. For example, it will be appreciated that a leukocyte deficiency, in the special, broad sense defined above, can arise in circumstances where an individual has acquired a disease, syndrome, disorder, condition or infection involving leukocyte dysfunction as well as in circumstances where an individual has acquired a disease, syndrome, disorder, condition or infection in which the endogenous leukocyte component is seemingly normal but in which alteration, augmentation or stimulation of the normal endogenous leukocyte activity is nevertheless indicated/required. In particular, a leukocyte deficiency as herein defined may be deemed to have arisen either as a result of a non-specific loss of T- and or B-cells, or as a result of a loss or deficiency of a particular T- and/or B-cell clonal population.

For convenience, such diseases, syndromes, disorders, conditions or infections are collectively defined herein as leukocytic deficiencies.

The processes of the invention are employed to create a leukocyte composition (e.g. forming part of a leukocyte cell bank) from a blood sample from a healthy individual donor. Thus, the invention is used to create a cellular resource of healthy leukocytic tissue from an individual donor that can be restored to that donor individual should the individual acquire a leukocytic deficiency at a later date.

In such therapies (referred to herein as restorative autotransplantation), the invention exploits the fact that many leukocytic deficiencies occur as part of a temporal sequence of events (which may or may not be causally interrelated), so that the creation of a leukocyte cell bank at a point in time predating onset of the leukocytic deficiency constitutes a therapeutic resource which can later be used restoratively.

The concept of restorative autotransplantation described above can be applied to all healthy individuals, irrespective of factors that might serve as indicators of susceptibility to leukocytic deficiency (for example age, medical history, genetic background and lifestyle). However, it does permit the identification of a particular class of individuals for which the processes of the invention may be particularly advantageously applied, as described in more detail in section III (entitled “Selection of donor individuals”). Moreover, since the leukocyte deficiencies as defined above and treatable according to the invention by restorative autotransplantation embrace an enormous variety of known diseases, these are discussed in greater detail in the following section XII (entitled “Exemplary indications”).

### X. Exemplary Indications

As mentioned in the preceding section, the therapeutic and prophylactic uses of the invention encompass a very broad spectrum of diseases, syndromes, disorders, conditions and infections.

### Infections

The invention may find application in the treatment of various infections. In this case, the endogenous leukocyte activity may be normal (or responding normally) but its alteration, augmentation or stimulation is nevertheless desirable. In others (such as HIV infection) the endogenous leukocyte activity is dysfunctional as a direct consequence of infection.

Infections which may be treated or prevented according to the invention include bacterial, fungal or viral infections, or infections by any other organism e.g. a protozoan, nematode, insect or other parasite.

A preferred application is the treatment of AIDS as a result of HIV infection. Here, CD4⁺ cells can be collected from an individual when healthy or non-infected, and stored for subsequent transplantation into said individual when HIV infection manifests itself or when AIDS develops, or CD4⁺ cell count falls etc. Such a procedure may be attractive to an individual with a life-style likely to place them at risk from contracting HIV infection.

### Cancers, Leukaemias and Sarcomas

The invention may find application in the treatment and prophylaxis of various malignancies: in general, any malignant or pre-malignant condition, proliferative or hyper-proliferative condition or any disease arising or deriving from or associated with a functional or other disturbance or abnormality in the cells or tissues of the body. Therapy or prophylaxis of various forms of cancer represents a preferred embodiment of the invention, and the treatment or prophylaxis of any cancerous cells or tissues of the body is contemplated.

Thus, the invention is not limited to any one type of proliferative disease (e.g. leukaemias, lymphomas, carcinomas or sarcomas), nor is it restricted to specific oncogenes or tumour-suppressor gene epitopes such as ras, prostate-specific antigen 1 or prostate-specific antigen 2, her-2/new, myc, myb, fos, fas, retinoblastoma, p53 etc. or other tumour cell marker epitopes that are presented in an HLA class I antigen restricted fashion or other such way so as to be identifiable by a leukocyte. All cancers such as leukaemia, lymphoma, breast, stomach, colon, rectal, lung, liver, uterine, testicular, ovarian, prostate and brain tumours such as gliomas, astrocytomas and neuroblastomas, sarcomas such as rhabdomyosarcomas and fibrosarcomas are included for the therapy or prophylaxis by the present invention.

Thus, the present invention finds application in the treatment or prophylaxis of breast cancer, colon cancer, lung cancer and prostate cancer. It also finds application in the treatment or prophylaxis of cancers of the blood and lymphatic systems (including Hodgkin's Disease, leukemias, lymphomas, multiple myeloma, and Waldenstrom's disease), skin cancers (including malignant melanoma), cancers of the digestive tract (including head and neck cancers, cesophageal cancer, stomach cancer, cancer of the pancreas, liver cancer, colon and rectal cancer, anal cancer), cancers of the genital and urinary systems (including kidney cancer, bladder cancer, testis cancer, prostate cancer), cancers in women (including breast cancer, ovarian cancer, gynecological cancers and choriocarcinoma) as well as in brain, bone carcinoid, nasopharyngeal, retroperitoneal, thyroid and soft tissue tumours. It also finds application in the treatment or prophylaxis of cancers of unknown primary site.

### XI. Posology

Those skilled in the art will be readily able to determine the amount of leukocyte composition to be autotransplanted in the medical applications according to the invention.

It should be noted that as few as 0.01×10⁸ (e.g. 1-10×10⁸) mature lymphocytes (which can be derived from a single sample of approximately 100 ml of normal human blood) are sufficient to boost the immune system of a subject and hence may have a beneficial effect according to the autologous transplantation method of the invention. It should be noted that the removal of a unit of blood is commonplace with over three million units of blood being taken, for allografting, from individuals annually in the UK alone.

The leukocyte composition administered may be derived from a single blood sample, or may constitute a pool of leukocyte compositions derived from a plurality of different blood samples taken from a donor individual at different times. The leukocyte composition administered may constitute all or a fraction of the deposited material, but preferably constitutes only a fraction thereof in order that multiple dosing can be achieved, optionally following cellular expansion of the residue (for example, T cell numbers may be increased by in vitro expansion using standard methods).

In applications based on T-cell activity, the number of mature T-cells administered is at least 0.01×10⁸, more preferably at least 0.1×10⁸, more preferably at least 1×10⁸ (e.g. at least 1-10×10⁸). The preferred ranges are 0.01×10⁸ to 10¹⁰ mature T lymphocytes, such as 0.1×10⁸ to 10¹⁰, 1×10⁸ to 10¹⁰ or 1×10⁹ to 10¹⁰ mature T lymphocytes.

Thus, the mature T-cell sample acquired for autotransplantation is at least 0.01×10⁸, generally in the range of 10⁸-10¹⁰ CD3⁺ mature T-cells, preferably 2×10⁸-10¹⁰, more preferably 3×10⁸-10¹⁰ CD3⁺ and even more preferably 4-5×10⁸-10¹⁰ CD3⁺ mature T-cells.

Conveniently, each sample prepared for autotransplantation contains 3×10⁸ CD3⁺ mature T-cells, more preferably 5×10⁸ and even more preferably 1×10⁹ CD3⁺ mature T-cells. If sufficient resources of blood are available from an individual, even more preferably still 4-5×10⁹ CD3⁺ mature T-cells or 10¹⁰ CD3⁺ mature T-cells may be used.

Preferably, the mature T-cell subpopulation sample acquired for autotransplantation which is CD3+ and CD8+ is at least 0.01×108, generally in the range of 0.25×108-0.25×1010, and more preferably 0.5×108-0.25×1010, and even more preferably 0.75×108-0.25×1010, and even more preferably still 0.75×108-0.25×1010 or 1.00-1.25×108-0.25×1010. Specific CD3+ and CD8+ cell numbers in each sample prepared for grafting is conveniently of the order of 0.2×108, preferably 0.4×108, or more preferably 1×108, or still more preferably 2×108, or more preferably 3×108, or more preferably 5×108. If sufficient resources from an individual are available, 1×109, preferably 2×109, 4×109 or more preferably 1×1010 CD3+ and CD8+ cells may be used.

Preferably, the mature T-cell subpopulation sample acquired for autologous transplantation which is CD3+ and CD4+ is at least 0.01×108, generally in the range of 0.1×108-0.5×1010, and more preferably 0.65×108-0.5×1010, and even more preferably 0.85×108-0.5×1010, and even more preferably still 1×108-0.5×1010 or 1.8-3.6×108-0.5×1010. Specific CD3+ and CD4+ cell numbers in each sample prepared for grafting is conveniently of the order of 0.2×1010, preferably 0.3×108, or more preferably 0.4×108, 0.5×108, 1×108, 2×108, 3×108, 4×108 or more preferably 5×108. If sufficient resources from an individual are available, 1×109, or more preferably 2×109, or more preferably 1×1010 CD3+ and CD4+ cells may be used.

Preferably, the mature T-cell natural killer subpopulation sample acquired for autotransplantation which is CD3+ and CD16/56+ is at least 0.01×108, generally in the range of 0.01×108-0.5×1010, preferably 0.02×108-0.5×1010, more preferably 0.03×108-0.5×1010, and even more preferably still 0.5×108-0.5×1010 or 0.5-2×108 to 0.5×1010. Specific CD3+ and CD16/56+ cell numbers in each sample prepared for grafting is conveniently of the order of 0.01×108, 0.2×108, 0.3×108, 0.5×108, 1×108, 2×108, 3×108, 5×108 or more preferably, if sufficient resources are available, 1×109, or more preferably 2×109, or more preferably 1×1010 CD3+ and CD16/56+ cells may be used.

In addition, the mature lymphocyte cell sample may preferably include B cells, such as CD19+ B lymphocytes. The mature B-cell sample included in the T-cell sample may be at least 107, 108 or 109, generally in the range of 107-1010 mature B-cells and preferably 2×107-1010 mature B-cells, more preferably 3×107-1010 mature B-cells, and even more preferably 4-5×107-1010 mature B-cells.

Specific numbers of B-cells in autograft is conveniently of the order of 3×107, preferably 5×108, more preferably 1×108 mature B-cells, and even more preferably still 4-5×109 or 1010 mature B-cells.

In addition, the lymphocyte cell sample may preferably include dendritic cells. The dendritic cell sample may be at least 107, 108 or 109 in number, and generally in the range of 107-1010 dendritic cells and preferably 2×107-1010 cells, more preferably 3×107-1010 cells, and even more preferably 4-5×107-1010 cells.

Specific numbers of dendritic cells in an autograft is conveniently of the order of 3×107, preferably 5×108, more preferably 1×108, and even more preferably still 4-5×109 or 1010 mature B-cells.

### XII. Stem Cell Amplification

As mentioned above, the invention finds application in processes for the collection and cryopreservation of stem cells. Such stem cells may then be used in various stem cell therapies, including the regeneration of the hematopoietic system in patients undergoing chemotherapy (e.g. in the treatment of leukaemia).

Such stem cells may be obtained from cord blood, peripheral blood (for example after treatment of the donor with mobilizing agents) or bone marrow.

In some cases, it may be desirable to amplify the stem cells prior to (or after) cryopreservation. Such a step may be indicated in circumstances where the absolute number of stem cells present in the specimen is too low for the intended therapeutic use. This commonly arises after harvesting of stem cells from necessarily small volumes of cord blood.

To facilitate this operation, the cryocyte bags of the invention may be gas permeable and provided with a culture compartment within which the stem cells can be contacted with the appropriate culture medium and/or growth factors and incubated to induce replication and thereby amplify the number of stem cells. The culture compartment is preferably releasably joined (for example with perforated heat sealed strips) to the rest of the cryocyte bag.

The culture compartment is preferably in fluid communication with the mixing compartment via a sealable conduit. It is conveniently provided with one or more media/growth factor ports.

Cryocyte bags provided with one or more culture compartments may also be used for the amplification of cell types other than stem cells. They may also be employed to effect treatments which do not involve cell multiplication, where the culture compartments simply acts as an incubation chamber. In such applications, the collected cells (such as T-lymphocytes, dendritic cells, stem cells etc.) may be contacted (and incubated, if necessary) with one or more active agents (such as adjunctive therapeutic factors, growth factors (including cytokines, lymphokines etc.), antigens, antibodies, markers, toxins, pyrogens, DNA, RNA, liposomes, vectors, viruses, other cells, cell lysates etc.) under controlled conditions and for predetermined time periods in order to activate or otherwise alter their developmental/therapeutic potential or biological activity. When used in this way, the culture compartment need not contain growth media of any kind, and may simply comprise a buffer system and the active agent(s). Thus, the culture compartment may be used for conducting any or all of the various leukocyte treatments described in Section V (above).

The provision of an integrated culture compartment/incubation chamber as described above permits cell manipulations that normally necessitate the opening of a closed system (and so require elaborate precautions against contamination) to be conducted within a single closed (or functionally closed) system.

### XIII. Exemplification

Embodiments of the present invention will now be described by way of example with reference to the accompanying drawings in which:

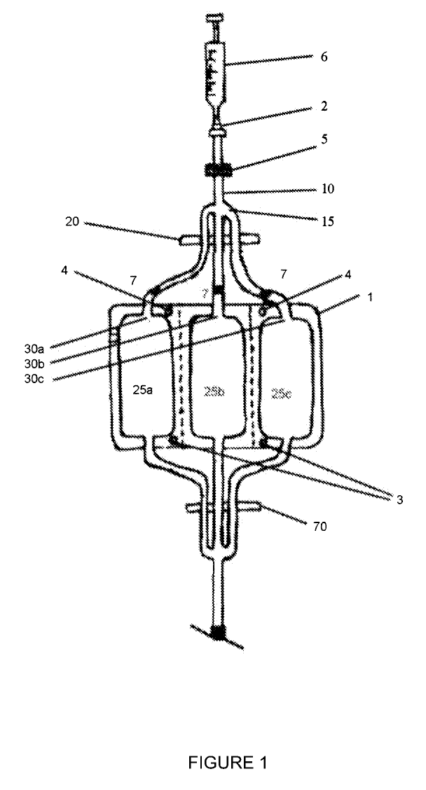

FIG. 1 is a schematic plan view of a first embodiment of the cryocyte bag of the invention showing the sterile introduction of cryoprotectant.

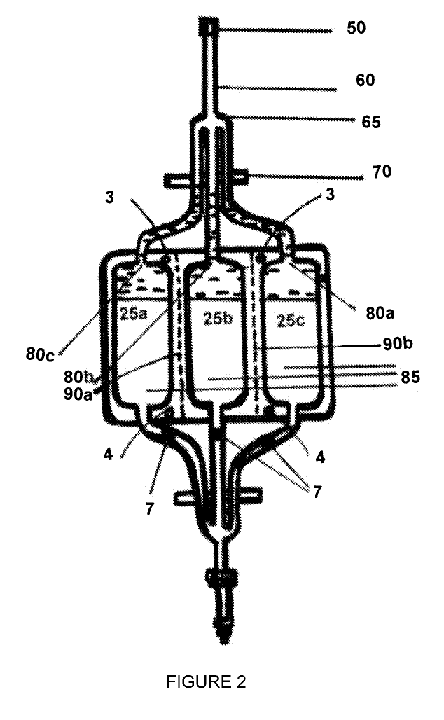

FIG. 2 is a schematic plan view of a first embodiment of the cryocyte bag of the invention showing the collection of leukocytes.

FIG. 3 is a schematic plan view of a second embodiment of the cryocyte bag of the invention.

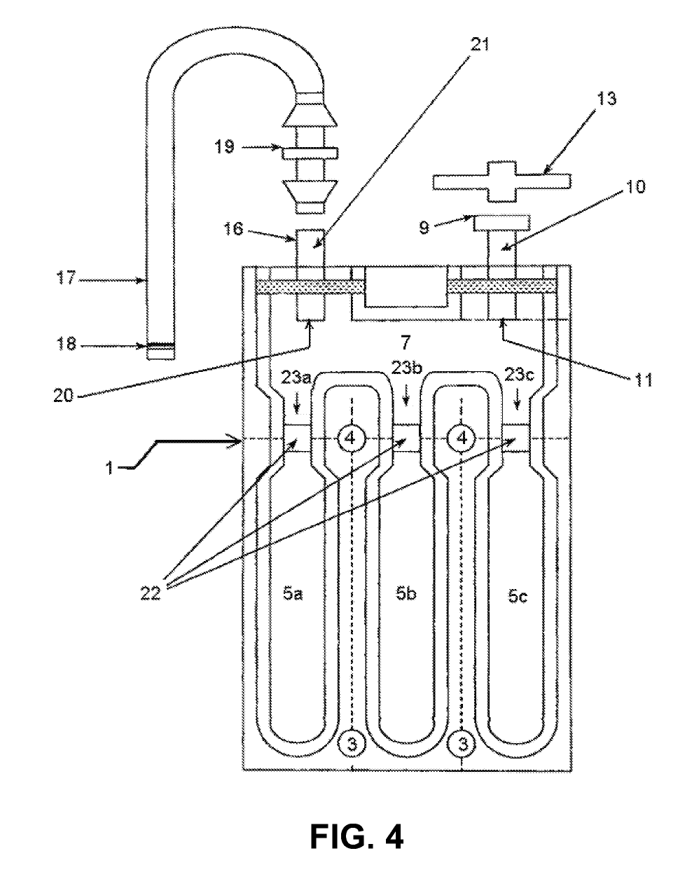

FIG. 4 is a schematic plan view of a third embodiment of the cryocyte bag of the invention.

Referring to FIG. 1, the leukocyte collect cryocyte bag 1 is suspended from a rack (not shown) by suspension holes 3. Clamp 20 is opened and cryoprotectant is introduced into the cryoprotectant inlet 2 and forced through submicron filter 5 by injection with a syringe 6. The cryoprotectant is pumped along the conduit 10, past the conduit manifold 15 and into each of three branches of the conduit, filling the dead space in the tubing and introducing three 1 ml aliquots into each of the leukocyte storage compartments 25a, 25b and 25c via cryoprotectant ports 30a, 30b and 30c, respectively. The cryoprotectant is then allowed to equilibrate before clamp 20 is closed. Each of the three conduit branches is then heat sealed at a point close to the ports 30a, 30b and 30c, so minimizing dead volume. The location of the heat seals are shown as 7 in FIGS. 1 and 2.

The bag 2 is then detached from the rack, inverted, and re-hung using suspension holes 4. At this stage, the cryoprotectant is contained within the bag and tubeset by the closed clamp 70 (as shown in FIG. 2).

Referring now to FIG. 2, inlet 50 is connected to an automated leukapheresis machine (not shown) and clamp 70 opened. A blood fraction highly enriched in leukocytes produced by the leukapheresis machine is then fed to the leukocyte inlet 50, along the conduit 60, past the conduit manifold 65 and into each of three branches of the leukocyte conduit. In this way three 4 ml aliquots are introduced into each of the leukocyte storage compartments 25a, 25b and 25c via leukocyte ports 80a, 80b and 80c, respectively. The conduit clamp 70 is then closed and the blood fractions and cryoprotectant allowed to mix and equilibrate. Each of the three conduit branches is then heat sealed at a point close to the ports 80a, 80b and 80c, so minimizing dead volume consistent with sufficient material for the subsequent removal of the leukocyte mixture under GMP conditions (e.g. by use of a sterile cannula).

The leukocyte storage compartments 25a, 25b and 25c, each now containing 4 ml of leukocytes mixed with 1 ml of cryoprotectant, are then separated by tearing along perforated heat sealed boundary strips 90a and 90b. Each of the three separate compartments 25a, 25b and 25c is then frozen and independently stored.

Referring now to a further embodiment of the invention (FIG. 3), cryocyte bag 1 is suspended from a rack (not shown) by suspension holes 3. Any residual air is expelled from the storage compartments 5a, 5b and 5c and mixing compartment 7 via submicron filter 13. Cryoprotectant is introduced into the cryoprotectant inlet 9 and forced along cryoprotectant conduit 10 through the cryoprotectant port 11 into mixing compartment 7 via submicron filter 13 by injection with a syringe (not shown). The cryoprotectant port is then closed by heat sealing. The location of the heat seal is shown by the solid black bar. The cryoprotectant conduit 10 is then severed.

Cell sample inlet 16 is connected to a COBE Spectra™ Automated Peripheral

Blood Stem Cell Set (AutoPBSC Set) in an automated leukapheresis machine (not shown). When valve 19 is opened a peripheral blood fraction highly enriched in CD34+ stem cells produced by the leukapheresis machine (not shown) can be fed to the sample port 17, along sample conduit 18 and into the mixing compartment 7. Once the required amount has been collected, the sample port is closed by heat sealing. The location of the heat seal is shown by the solid black bar. The sample conduit 18 is severed and the cryocyte bag can then be detached from the rest of the AutoPBSC tubing set (which set is then removed from the leukapheresis apparatus and discarded).

The sample and cryoprotectant are then thoroughly mixed and equilibrated by massaging the exterior walls of the mixing compartment 7. The cryocyte bag 1 is then detached from the rack, inverted, and re-hung using suspension holes 4. Three aliquots (of approximately equal volumes, though precise portioning is not necessary) of the cryoprotectant/sample mixture are then allowed to flow into each of the storage compartments 5a, 5b and 5c via mix conduits 23a, 23b and 23c. Once the entire volume of sample/cryoprotectant mixture in the mixing compartment 7 has drained into the storage compartments 5a, 5b and 5c, each of the mix conduits 23a, 23b and 23c are heat sealed at the locations shown by the three solid black bars.

It should be noted that the cryocyte bag shown in FIG. 3 can be filled by an alternative procedure wherein the peripheral blood fraction is introduced into the mixing compartment before the cryoprotectant. It will be understood that the order in which the cryoprotectant and cell sample are introduced is not important: all that matters is that the sample/cryoprotectant are thoroughly mixed and equilibrated prior to distribution to the storage compartments.

The filled cell storage compartments 5a, 5b and 5c, each now containing CD34+ stem cells mixed and equilibrated with cryoprotectant, are then separated from each other and from the empty mixing compartment 7 by tearing along perforated heat sealed boundary strips indicated by the dashed lines. Each of the three separate compartments 5a, 5b and 5c is then frozen and independently stored.

Referring now to a further embodiment of the invention (FIG. 4), cryocyte bag 1 is suspended from a rack (not shown) by suspension holes 3. Any residual air is expelled from the storage compartments 5a, 5b and 5c and mixing compartment 7 via submicron filter 13.

Sterile input tube 17 (2 inch pvc with end seal 18) is connected to the cell sample inlet 16 of the cryocyte bag and the bag then connected to the output tube of a COBE Spectra™ Automated Peripheral Blood Stem Cell Set (AutoPBSC Set) in an automated leukapheresis machine (not shown) after breaking seal 18. When valve 19 is opened a peripheral blood fraction highly enriched in CD34+ stem cells produced by the leukapheresis machine (not shown) can be fed to the sample port 20, along sample conduit 21 and into the mixing compartment 7. Once the required amount (about 5 ml) has been collected, the sample port is closed by heat sealing. The location of the heat seal is shown by the stippled bar. The sample conduit 21 is severed and the cryocyte bag can then be detached from the rest of the AutoPBSC tubing set (which set is then removed from the leukapheresis apparatus and discarded). Alternatively, the input tube 17 can be heat sealed near inlet 16.

The cryocyte bag containing the cell sample is then weighed using a second, empty bag as tare. The weight/volume of the sample is then calculated and the volume of cryoprotectant required determined. The required volume of cryoprotectant is then introduced into the cryoprotectant inlet 9 and forced along cryoprotectant conduit 10 through the cryoprotectant port 11 into mixing compartment 7 via submicron (0.22 micron) filter 13 by injection with a syringe (not shown). The cryoprotectant port is then closed by heat sealing. The location of the heat seal is shown by the stippled bar. The cryoprotectant conduit 10 is then severed.

The sample and cryoprotectant are then thoroughly mixed and equilibrated by massaging the exterior walls of the mixing compartment 7. The cryocyte bag 1 is then detached from the rack, inverted, and re-hung using suspension holes 4. Three aliquots (of approximately equal volumes, though precise portioning is not necessary) of the cryoprotectant/sample mixture are then allowed to flow (and/or forced by additional squeezing of the mixing compartment) into each of the storage compartments 5a, 5b and 5c via mix conduits 23a, 23b and 23c.

Once the entire volume of sample/cryoprotectant mixture in the mixing compartment 7 has been drained and/or squeezed into the storage compartments 5a, 5b and 5c, the storage compartments should be full and free of air bubbles and each of the mix conduits 23a, 23b and 23c are heat sealed and the mixing compartment detached. Two heat seals may be used so that the empty mixing compartment 7 can be detached in a sealed state by severing between the two seals.

Located in the isthmuses between the storage compartments 5a, 5b and 5c and the mixing compartment 7 are three lengths of 4 mm TFE tubing 22. These function as patency tubes, and serve to maintain mix conduits 23a, 23b and 23c in an open (or patent) state during and after storage. In the absence of the patent tubes, surface tension forces acting on the walls of the transfer conduits may occlude them and make subsequent retrieval of the samples from the storage compartments via the conduits after storage (when the mix conduits act as sample retrieval conduits) difficult.

The filled cell storage compartments 5a, 5b and 5c, each now containing CD34+ stem cells mixed and equilibrated with cryoprotectant, are then separated from each other. Each of the three separate compartments 5a, 5b and 5c is then frozen and independently stored. After storage and thawing, the mix conduits 23a, 23b and 23c serve as sample retrieval ports, and are maintained in a patent state by the patent tubes 22 to facilitate sample recovery (e.g. by pipette or cannula).

### XIV. EQUIVALENTS

The foregoing description details presently preferred embodiments of the present invention which are therefore to be considered in all respects as illustrative and not restrictive. Those skilled in the art will recognize, or be able to ascertain, using no more than routine experimentation, many equivalents, modifications and variations to the specific embodiments of the invention described specifically herein. Such equivalents, modifications and variations are intended to be (or are) encompassed in the scope of the following claims.

1. An apheresis tubing set comprising a cryocyte bag for collecting cells separated during apheresis.

2. The tubing set of claim 1 wherein the cryocyte bag comprises a mixing compartment in fluid communication with a cell storage compartment, wherein the mixing compartment comprises a cryoprotectant port and a cell sample port and wherein the storage and mixing compartments are in fluid communication via a mix conduit.

3. The tubing set of claim 2 wherein the compartments and mix conduit are defined by seals (e.g. heat seals) within the cryocyte bag.

4. The tubing set of any one of the preceding claims wherein the cryoprotectant port is connected to a cryoprotectant inlet by a cryoprotectant conduit.

5. The tubing set of any one of the preceding claims wherein the cryoprotectant port is separated from the cryoprotectant inlet by a sterile barrier filter within the cryoprotectant conduit.

6. The tubing set of any one of the preceding claims wherein the cryocyte bag comprises two or more independent cell storage compartments for collecting two or more aliquots of the cells.

7. The tubing set of claim 6 wherein the cryocyte bag comprises three or more independent cell storage compartments for collecting three or more aliquots of the cells.

8. The tubing set of claim 6 or claim 7 wherein each of the independent cell storage compartments is in fluid communication via a mix conduit with a single mixing compartment.

9. The tubing set of any one of the preceding claims wherein the mix conduit(s):
(a) are sealable (for example, heat sealable); and/or
(b) comprise patency tube(s).

10. The tubing set of any one of the preceding claims wherein the cryocyte bag is of thermoplastic material.

11. The tubing set of any one of the preceding claims wherein the cryocyte bag is of a fluorocarbon resin.

12. The tubing set of claim 11 wherein the fluorocarbon resin is PTFE (e.g. Teflon®), PFA, FEP, ETFE, ECTFE, PVF or CTFE.

13. The tubing set of any one of the preceding claims wherein the mixing and storage compartment(s) are releasably joined.

14. The tubing set of any one of the preceding claims wherein the cryocyte bag is tailed with thermoplastic tubing.

15. The tubing set of claim 14 wherein the thermoplastic tubing tail(s) communicate with the cryoprotectant port and/or the cell sample port.

16. The tubing set of claim 14 or claim 15 wherein the thermoplastic tail(s) are of PVC.

17. The tubing set of any one of the preceding claims wherein at least some components (e.g. the vessel(s) and/or interconnecting tube(s)) of the tubing set other than the cryocyte bag are of PVC.

18. The tubing set of any one of the preceding claims which is a leukapheresis tubing set.

19. The tubing set of any one of the preceding claims which is closed or functionally closed.

20. The tubing set of claim 1 which is a closed (or functionally closed) apheresis (e.g. leukapheresis) tubing set comprising a cell (e.g. leukocyte) collect bag having two or more independent cell (e.g. leukocyte) storage compartments.

21. The tubing set of claim 20 wherein the collect bag is a leukocyte collect bag having three leukocyte storage compartments.

22. The tubing set of claim 20 or claim 21 wherein the storage compartments are releasably joined.

23. The tubing set of any one of the preceding claims wherein the compartments (e.g. the holding compartments) are releasably joined by at least one breakable bridge.

24. The tubing set of claim 23 wherein the at least one breakable bridge comprises a perforated or scored connecting strip.

25. The tubing set of any one of claims 20 to 24 wherein the collect bag comprises bipolar suspension means.

26. The tubing set of any one of the claims 20 to 25 wherein each of the storage compartments is provided with a cryoprotectant port and a leukocyte port.

27. The tubing set of claim 26 wherein the cryoprotectant port is connected to a cryoprotectant inlet by a conduit.

28. The tubing set of claim 27 wherein the cryoprotectant port is separated from the cryoprotectant inlet by a sterile barrier filter.

29. The tubing set of claim 27 wherein the conduit branches into the cryoprotectant port of each of the storage compartments at a manifold.

30. The tubing set of claim 28 wherein a single sterile barrier filter is located between the cryoprotectant inlet and the manifold.

31. The tubing set of claim 30 wherein the cryoprotectant ports can be sealed, for example by clamping of the conduit.

32. The tubing set of any one of claims 27 to 31 wherein the leukocyte port is connected to a leukocyte inlet by a conduit.

33. The tubing set of claim 32 wherein the conduit branches into the leukocyte port of each of the storage compartments at a manifold.

34. The tubing set of claim 33 wherein the leukocyte ports can be sealed, for example by clamping of the conduit.

35. The tubing set of any one of the preceding claims further comprising a blood processing vessel.

36. The tubing set of claim 35 wherein the blood processing vessel comprises: (a) a centrifuge loop; or (b) a Latham bowl; or (c) a filtration device.

37. The tubing set of any one of the preceding claims further comprising a sample vessel for holding an isolated tissue (e.g. peripheral blood, cord blood or bone marrow) sample.

38. The tubing set of any one of the preceding claims further comprising means (e.g. comprising a needle) for collecting an isolated blood or bone marrow sample from an individual.

39. The tubing set of any one of the preceding claims which is adapted for use in an automated leukapheresis device.

40. A cryocyte or leukocyte collect bag for use with the tubing set as defined in any one of the preceding claims.

41. Apparatus for selectively separating and removing: (a) cells (e.g. leukocytes or stem cells), comprising an apheresis (e.g. leukapheresis) device loaded with the tubing set of any one of claims 1 to 39; or (b) bone marrow components, comprising an apheresis device loaded with the tubing set of any one of claims 1 to 39.

42. The apparatus of claim 41 wherein the apheresis device comprises: (a) a separation device (e.g. a centrifuge rotor or filter) and (b) one or more pumps for conveying the sample through the tubing set.

43. A process for producing a cell composition (e.g. a leukocyte composition suitable for CAT therapy) comprising the step of selectively separating and collecting cells using the leukapheresis apparatus of claim 41 or claim 42, optionally wherein the process is conducted under closed (or functionally closed) conditions).

44. A composition (e.g. leukocyte composition) obtainable (or obtained) by the process of claims 43.

45. The composition of claim 44 for use in therapy or prophylaxis (e.g. for CAT therapy).

46. Use of the composition of claim 44 for the manufacture of a medicament for use in therapy, e.g. in autotransplantation.

47. A process for producing a cell (e.g. leukocyte) bank, optionally under closed or functionally closed conditions, comprising the steps of:
(a) selectively separating and collecting cells using the apparatus of claim 41 or claim 42;
(b) rendering the cells dormant (e.g. by cryogenic preservation); and
(c) applying steps (a) and (b) iteratively to a series of tissue (e.g. blood or bone marrow) samples from different healthy donor individuals.

48. The process of claim 47 further comprising the step of retrievably depositing the cells into two or more independent storage systems to produce a cell (e.g. leukocyte) bank which exhibits deposit redundancy.

49. The process of claim 47 or claim 48 further comprising the step of digitally storing information obtained from each donor individual in a digital information unit so as to permit matching of deposit and donor for later autologous transplantation.

50. A cell (e.g. leukocyte) bank obtainable (or obtained) by the process of any one of claims 47 to 49.

51. A cryocyte bag for collecting cells separated during apheresis comprising one or more cell storage compartment(s) having a sample transfer port, wherein the sample transfer port comprises a patency tube.
